(19) United States
(12) Patent Application Publication
(10) Pub. No.: US 2006/0229632 A1
(43) Pub. Date: Oct. 12, 2006

(54) Handpiece tip

(75) Inventors: Sean C. Madden, Mission Viejo, CA (US); John M. Bourne, Torrance, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/100,853

(22) Filed: Apr. 7, 2005

(51) Int. Cl. A61F 9/00

(52) U.S. Cl. 606/107

(57) ABSTRACT

A tip for a surgical handpiece having two coaxial tubes or channels mounted within a body. The tip has one or more features that shape the fluid discharge so as to optimize the performance of the handpiece for varying parts of the surgical procedure.

### BACKGROUND OF THE INVENTION

This invention relates generally to the field of cataract surgery and more particularly to a handpiece tip for practicing the liquefracture technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted concurrently with the injection of the heated solution and the injection of a relatively cool solution, thereby quickly cooling and removing the heated solution. This technique is more fully described in U.S. Pat. No. 5,616,120 (Andrew, et al.), the entire content of which is incorporated herein by reference. The apparatus disclosed in the publication, however, heats the solution separately from the surgical handpiece. Temperature control of the heated solution can be difficult because the fluid tubes feeding the handpiece typically are up to two meters long, and the heated solution can cool considerably as it travels down the length of the tube.

U.S. Pat. No. 5,885,243 (Capetan, et al.) discloses a handpiece having a separate pumping mechanism and resistive heating element. Such a structure adds unnecessary complexity to the handpiece.

U.S. Pat. No. 6,579,270 B2 (Sussman, et al.) discloses a surgical handpiece and tip having two coaxial tubes or channels mounted within a body. The first tube is used for aspiration and is smaller in diameter than the second tube so as to create an annular passage between the first and second tube. The annular passage communicates with a pumping chamber formed between two electrodes. The pumping chamber works by boiling a small volume of the surgical fluid. As the fluid boils, it expands rapidly, thereby propelling the liquid downstream of the pumping chamber out of the annular passage. The distal end of the annular gap is sealed by a nozzle at the distal ends of the first and second tube and a plurality of orifices or ports may be formed in the nozzle. As the expanding gas is propelled down the annular gap, the gas/liquid stream is forced out of the distal orifice in a controlled and directed manner. However, aspiration and irrigation flow patterns different that those described in this patent are sometimes desired, such as during cortical clean up or posterior capsule washing or lavage.

Therefore, a need continues to exist for a simple surgical handpiece and tip that can heat internally the solution used to perform the liquefracture technique.

### BRIEF SUMMARY OF THE INVENTION

The present invention improves upon the prior art by providing a tip for a surgical handpiece having two coaxial tubes or channels mounted within a body. The tip has one or more features that shape the fluid discharge so as to optimize the performance of the handpiece for varying parts of the surgical procedure.

Accordingly, one objective of the present invention is to provide a tip for a surgical handpiece having at least two coaxial tubes.

Another objective of the present invention is to provide a tip for a handpiece having a pumping chamber.

Another objective of the present invention is to provide a tip for a surgical handpiece having a device for delivering the surgical fluid through the handpiece in pulses.

Still another objective of the present invention is to provide a tip for a handpiece that delivers fluid pulses in a controlled and directed manner.

These and other advantages and objectives of the present invention will become apparent from the detailed description and claims that follow.

### BRIEF DESCRIPTION OF THE DRAWINGS

FIG. 1 is a partial front, upper left perspective view of the handpiece tip of the present invention.

FIG. 2 is a partial cross-sectional view of the handpiece tip of the present invention.

FIG. 3 is a front elevational view of the handpiece tip of the present invention.

FIG. 4 is a partial cross-sectional view of the handpiece tip of the present invention.

### DETAILED DESCRIPTION OF THE INVENTION

Handpieces suitable for use with the present invention include the INFINITI® AQUALASE® surgical system which is commercially available from Alcon Laboratories, Inc., Fort Worth, Tex. This system uses a tip generally described in FIG. 23 and 24 and column 7, lines 33-45 of U.S. Pat. No. 6,579,270 B2 (Sussman, et al.) and these portions of such patent are specifically incorporated herein by reference. As described in this patent, tip 900 may alternatively consist of outer tube 965 surrounding and coaxial with inner tube 967. Distal tip 902 of outer tube 965 is flared or belled so as to allow nozzle 905 to be inserted between outer tube 965 and inner tube 967. As best seen in FIG. 23, nozzle 905 contains fluid channel 907 that communicates with orifice 904. Nozzle 905 seals annular gap 969 between outer tube 965 and inner tube 967. Pressurized fluid flowing down annular gap 969 is forced into fluid channel 907 and out orifice 904.

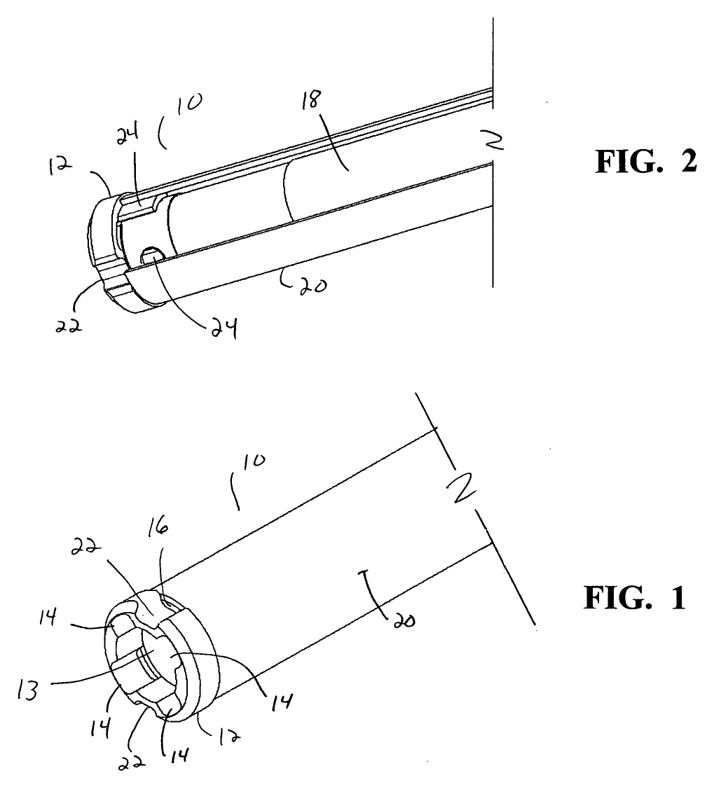

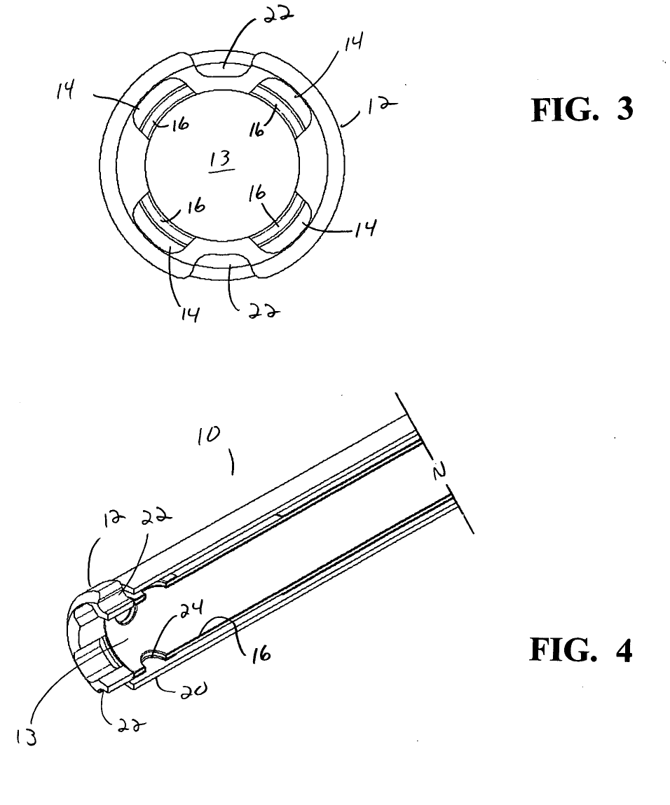

As best seen in FIGS. 1-4, tip 10 of the present invention generally includes rounded tip cap or body 12 having a generally ring-like shape with central opening 13 and a plurality of injection ports 14. Tip cap 12 contains extension 24 that generally seals annular gap 16 between inner tube 18 and outer tube 20 and helps to maintain the concentricity of inner tube 18 and outer tube 20, but cap 12 allows aspiration through central opening 13 and inner tube 18. Cap 12 contains a plurality of integrally formed, inwardly facing injection ports 14 that are in fluid communication with annual gap 16. Such a construction allows pulse of fluid being propelled down annular gap 16 to exit gap 16 through ports 14. The shape and size of ports 14 may be constructed so as to focus the fluid pulses as they exit ports 14. Cap 12 also contains a plurality of aspiration bypass ports 22, also in fluid communication with gap 16. Ports 22 face outwardly, away from central opening 13; allowing fluid to flow into gap 16 and into inner tube 18 through ports 24 in the event that opening 13 becomes clogged or occluded.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with ultrasonic and/or rotating cutting tips to enhance performance.

1. A tip for a handpiece, comprising:
a) an inner tube mounted within an outer tube so as to form an annular gap between the inner tube and the outer tube, the inner tube allowing aspiration and the gap allowing irrigation;
b) a tip cap having a central opening, the tip cap sealing the annual gap at the distal ends of the inner tube and the outer tube, the tip cap having a plurality of injection ports in fluid communication with the annular gap, and a plurality of aspiration bypass ports in fluid communication with the inner tube, the bypass ports directed away from the central opening; and
c) a port in the inner tube providing fluid communication between the inner tube and the annular gap.

2. The handpiece tip of claim 1 wherein the injection ports are directed toward the central opening.
